(19) United States
(12) Patent Application Publication
(10) Pub. No.: US 2017/0108488 A1
(43) Pub. Date: Apr. 20, 2017

(54) Array and Method of Use

(71) Applicant: Quick Biology, Inc., Arcadia, CA (US)

(72) Inventors: Yunguang TONG, Los Angeles, CA (US); Hei CHAN, Rosemead, CA (US); Chen QING, Kunming (CN); Jian DING, Shanghai (CN)

(21) Appl. No.: 14/995,499

(22) Filed: Jan. 14, 2016

Related U.S. Application Data
(60) Provisional application No. 62/242,782, filed on Oct. 16, 2015.

(51) Int. Cl. G01N 33/50; C12Q 1/68

(52) U.S. Cl. CPC G01N 33/5011; C12Q 1/6886; C12Q 2600/136; C12Q 2600/178

(57) ABSTRACT

A method of identifying drug-drug, gene-drug or gene-gene interaction includes providing a plurality of arrays, each array including a plurality of ready-to-use plate wells, and each well including a bioactive material to target one or more cell component; adding a control and a candidate agent into the wells to form a control-material mix and an agent-material mix, respectively; when the control and the candidate agent are not cells, culturing a predetermined number of cells according to the number of the arrays and suspending and plating the cells into the arrays, when the control and the candidate agent are cells, no additional cells are needed; incubating the arrays in a cell culture incubator; measuring a predetermined signal; and collecting and analyzing data, thereby identifying the drug-drug, gene-drug, or gene-gene interaction.

### FIELD OF THE INVENTION

This invention relates to arrays and methods of use thereof, for identifying drug-drug, gene-drug and gene-gene interactions.

### BACKGROUND OF THE INVENTION

The significance of drug-drug, drug-gene and gene-gene interactions have gained increasing recognition. Strategies to reduce drug interactions, to optimize the therapy in clinical practice lag behind the initiatives taken during the drug development and approval process to predict and confirm drug interactions. The conventional methods of drug interaction and drug-gene studies are costly and time consuming. Predicted interactions do not always lead to discernibly toxicity or therapeutic failure.

Therefore, there is a need to develop a fast and cost effective method to identify drug-drug and gene-drug interactions.

### SUMMARY OF THE INVENTION

The invention provides an array comprising a plurality of ready-to-use plate wells. The cells of interest are plated into the wells and mixed with material of interest, incubated for a set time and measured for predetermined signals. The materials include chemicals, drugs, siRNA, miRNA, growth factors, hormones, proteins and any other bioactive agents. The arrays are designed in a way to cancel out variations, for example, a mirror/rotational symmetry.

The invention further provides a method of identifying drug-drug, gene-drug and gene-gene interactions in the array. The method includes the steps of culturing a predetermined number of cells; adding the candidate gene and related control into the array to form control-materials mix and agent-materials mix; suspending and plating cells into the array; incubating the array in cell culture incubator; measuring the predetermined signal; and collecting and analyzing data, thereby identifying drug-drug, gene-drug and gene-gene interactions.

In one embodiment, the invention provides a drug array comprising a plurality of ready-to-use plate wells wherein cells of interest are plated into the wells and mixed with candidate drugs of interest, incubated for a set time and measured for viable cell numbers. In certain embodiments, the arrays are designed in a mirror symmetry. In certain embodiments, each drug is provided at two concentrations that are optimized to block the activities of the drug target in the cells.

The present invention provides the method of identifying drug-drug and gene-drug interactions in the drug array. In certain embodiments, the drug array is used to investigation of the mechanism of action of a gene or drug candidate. In certain embodiment, the drug array is used to survey the interaction of a gene or drug candidate. In certain embodiment, the drug array is used to determining pathways that render cancer cells sensitive or resistant to a given drug. In certain embodiment, the drug array is used to determining drug interactions for improved therapy.

In another embodiment, the invention provides a siRNA array comprising a plurality of ready-to-use well plate wherein siRNA is provided in each well, transfection mixture and cells of interest are plated into the wells, incubated for a set time and measured for viable cell numbers.

The present invention provides the method of identifying gene-drug and gene-gene interactions in the siRNA array. In certain embodiments, the siRNA array is used to screen genes regulating anti-cancer drug resistance.

### BRIEF DESCRIPTION OF THE DRAWINGS

The drawings set forth herein are illustrative of embodiments of the invention and are not limit the scope of the invention as encompassed by the claims.

FIG. 1 illustrates an exemplary drug array for identifying drug-gene interaction.

FIG. 2 illustrates an exemplary drug array for identifying gene-gene interaction using stable cell lines.

FIG. 3 illustrates an exemplary drug array for identifying gene-gene interaction using transient transfection.

FIG. 4 provides an experimental result showing effect of combined drug treatment on cell viability experiment.

FIG. 5 provides an easier visualization of the differences in cell viability by inhibition rates.

FIG. 6 provides an easier visualization of the differences in inhibition rates.

FIG. 7 illustrates an exemplary siRNA array for identifying drug-gene interaction.

FIG. 8 illustrates and exemplary siRNA array for identifying gene-gene interaction using stable knockdown/over-expression cell lines.

FIG. 9 illustrates an exemplary siRNA array for identifying gene-gene interaction using transient knockdown.

FIG. 10 provides an exemplary visualization of the effect of siRNA transfection on cell viability.

FIG. 11 provides an easier visualization of the differences in cell viability by inhibition rates.

FIG. 12 provides an exemplary visualization of the effect of siRNA transfection on cell viability by difference in inhibition rates of pre-treated and control samples.

### DETAILED DESCRIPTION OF THE INVENTION

The present invention provides arrays to identify drug-drug, drug-gene, and gene-gene interactions. The invention provides an array comprising a plurality of ready-to-use plate wells. The cells of interest are plated into the wells and mixed with material of interest, incubated for a set time and measured for predetermined signals. The materials include chemicals, drugs, siRNA, miRNA, growth factors, hormones, proteins and any other bioactive agents. The arrays are designed in a way to cancel out variations, for example, a mirror/rotational symmetry.

The invention further provides a method of identifying drug-drug, gene-drug and gene-gene interactions in the array. The method includes the steps of culturing a predetermined number of cells; adding the candidate gene and related control into the array to form control-materials mix and agent-materials mix; suspending and plating cells into the array; incubating the array in cell culture incubator; measuring the predetermined signal; and collecting and analyzing data, thereby identifying drug-drug, gene-drug and gene-gene interactions.

As used herein, the term “array” refers to a collection of materials displayed on a solid surface, usually a glass or plastic chip, and being used to study drug-drug, drug-gene and gene-gene interactions.

Various publications, including patents, published applications, technical articles and scholarly journals are cited throughout the specification. Each of these cited publications is incorporated by reference herein, in its entirety.

### Drug Array

In one embodiment, the present invention provides a drug array that uses highly selective chemicals as tools to investigate the mechanisms of action of a gene or a drug. The arrays can be used to survey the interaction of a candidate gene or chemical.

In further embodiments, each chemical in the drug array targets a specific target in a critical pathway, which may contribute to drug resistance or insensitivity. One of the major implications could be determining which pathways may render cancer cells sensitive or resistant to a given drug. Based on the outcome of the drug array assay, drug-drug interactions could be identified and further investigated for improved therapy.

The drug array of the present invention further provides a method to survey gene-drug interactions, such as synthetic lethality. One example of gene-drug interaction is BRCA1 and PARP inhibitors. BRCA1 is a tumor suppressor protein which is important for DNA repair. Tumors with BRCA1 mutation are deficient in double strand break repair and accumulation of such lesions renders the cells unviable. PARP inhibitors take advantage of this weakness by increasing single strand breaks in DNA, which form double strand breaks during replication [1]. Similarly, KRAS mutations were found to cause resistance to anti-EGFR therapies in colorectal cancer (CRC) [2]. Inhibition of other target genes such as PLK1, APC/C, TBK1, TAK1, STK33, and GATA2 can induce enhanced cell death in KRAS mutant tumors [3], which provides a valuable strategy to overcome anti-EGFR therapy resistance in CRC patients. In addition to these distinguished examples, an increasing number of drug-drug and gene-drug interactions are observed and published in high impact journals.

Studies have been done on MEK inhibitor (GSK1120212) &CDK4 Inhibitors (PD-0332991) in SB-2 cell line. Melanomas with a mutant NRAS oncogene have no effective targeted therapy. Research indicated that CDK4 protein as the key effector for tumor progression. PD-0332991, a selective dual inhibitor of CDK4 and 6, was then used in combination with the MEK inhibitor GSK1120212 to mimic the effects of NRAS^{Q61K} extinction and tumor regression was achieved in SB-2 tumor xenograft [4].

Studies have also been done on RAF Inhibitors (PLX4032) and EGFR inhibitor (BIBW2992) in HTC-C3 cell line. The BRAF oncogene has activating mutations in some cancers, and the inhibitor PLX4032 exhibits a high success rate in melanomas but not in colorectal cancers. A kinase screening assay using a library of 535 shRNAs revealed a potential for the inhibition of EGFR to synergize with BRAF inhibition. Further investigation showed that the inhibition of BRAF causes strong feedback activation of EGFR, leading to poor therapeutic efficacy in colorectal cancers with BRAF mutations. This provides a strong rationale for the combined use of BRAF and EGFR inhibitors. Moreover, the combined use of BRAF and EGFR inhibitors induced apoptosis, but either drug alone could not produce the same effect. This discovery was made possible through a large kinase screening and validation project which required significant costs and efforts [5].

Studies have also been done on PARP inhibitor (AZD2281) and EWS-FLI1 siRNA in A673 cell line. Targeted cancer therapy, such as the inhibitor of the BCR-ABL translocation gene product, has been a major breakthrough in the field and the mechanism of action has been well studied. Although there are a number of cancer drugs in the clinic and under investigation, the responsiveness is widely variable and the mechanisms are poorly understood. There is also a lack of biomarkers to predict their therapeutic effectiveness. To find new biomarkers for these cancer drugs, a large scale screening study was carried out and 48,178 drug-cell-line combinations were assayed. Most of the cancer genes and drugs had at least one interaction associated with sensitivity or resistance. The EWS-FLI1 rearrangement is an example of one such biomarker for sensitivity to PARP inhibitors in Ewing's sarcoma cells [6].

In one embodiment of the present invention, 66 chemicals in 3 arrays are provided in Tables 1-3 for selectively targeting 66 primary targets. The targets of these chemicals are well defined and the chemicals are well characterized. Therefore, through the drug array platform, the effects of the drug combination and mechanisms of gene-drug interactions can be interrogated at a much lower cost and effort.

### Methods

I. General Design

Each drug array plate is designed to compare two different samples in parallel on one ready-to-use 96-well plate. To better observe the drug-drug and gene-drug interactions, each drug is provided at two concentrations (0.5 μM and 2.5 μM). This concentration range has been optimized to block the activity of the drug target in most cell lines. The arrays are designed in a mirror symmetry layout to cancel out the edge effects.

II. Preparation of the Experiments

Determine Cell Number

The cell culture conditions, such as the number of the cells, can significantly affect the results and need to be empirically determined before the experiment. In general, cells in control wells should be seeded at densities that could reach optimal population densities (80-90% confluence) at the end of experiments (48-72 hours). Most cancer cells require 3,000-8,000 cells per well in 96 well plates.

The right number of cells is critical to the success of the assay. Too many cells will cause over-confluence and deplete the nutrition of the medium before the end of the assay. Too few cells will lead to large variation and increased edge effects.

Determine Drug Concentration for Drug-Drug Interaction

To observe potential drug-drug interactions, the user-provided drug should be added at a concentration around IC15-IC30 (a concentration that causes 15%-30% inhibition of cell proliferation). Concentrations higher than IC50 will likely saturate the assay and decrease the sensitivity of the array.

Determine Time Points for Transient Transfection

To observe potential gene-drug interaction, it is preferred to generate isogenic cell lines of target genes, for example, stable knockdown/over-expression cell line vs. parental control cell line. Transient transfection may be used as long as the transfection efficiency is high (>70%). The effect of over-expression or knock-down of transient transfection usually last for 7 days. The gene-drug interaction experiment should be performed 24-48 hours after transfection.

### Experimental Procedure

### I. Seeding Cells

### Experiment Design A: Drug-Drug Interaction Study

Culture a sufficient number of cells according to the number of arrays needed. Add the candidate chemical into the drug array to form vehicle control-drug mix and drug-drug mix. 100 ul suspended cells are plated into the drug array according to the diagram below. Incubate the drug array in a cell culture incubator for 48-72 hours.

Alternative option for adherent cells is to plate a pre-determined number of cells in a user-provided culture plate. Incubate for 24 hours after plating, and then add the candidate chemical and vehicle control to the Drug Array plate and mix to form the drug-drug combinations. Transfer the drug-drug mixtures to the culture plate containing the cells.

### Experiment Design B: Gene-Drug Interaction Study Using Stable Cell Lines

Culture the stable knockdown/over-expression cells and parent control cells, and then resuspend and seed the cells into the drug array. Incubate the drug array in a cell culture incubator for 48-72 hours.

Alternative option for adherent cells is to plate a pre-determined number of cells in a user-provided culture plate. Incubate for 24 hours after plating, transfer the drug-drug mixtures to the culture plate containing the cells.

### Experiment Design C: Gene Drug Interaction Study Using Transient Transfection

Transient transfection maybe used as long as high transfection efficiency (>80%) is reached. The over-expression or knock-down effect of transient transfection usually lasts for 7 days. Perform transient transfection and incubate cells for 24 hours. Resuspend cells and seed into the drug array. Incubate the drug array in a cell culture incubator for 48-72 hours.

Alternative option for adherent cells is to plate a pre-determined number of cells in a user-provided culture plate. Incubate for 24 hours after plating, transfer the drug-drug mixtures to the culture plate containing the cells.

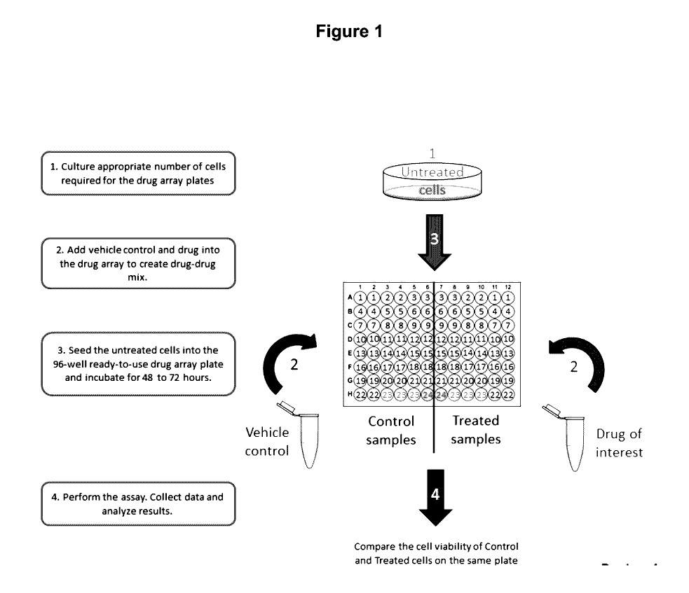

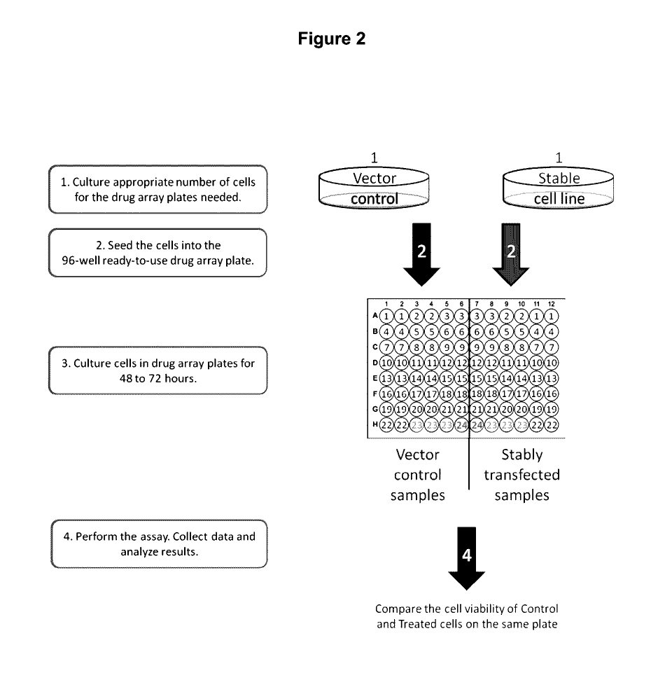

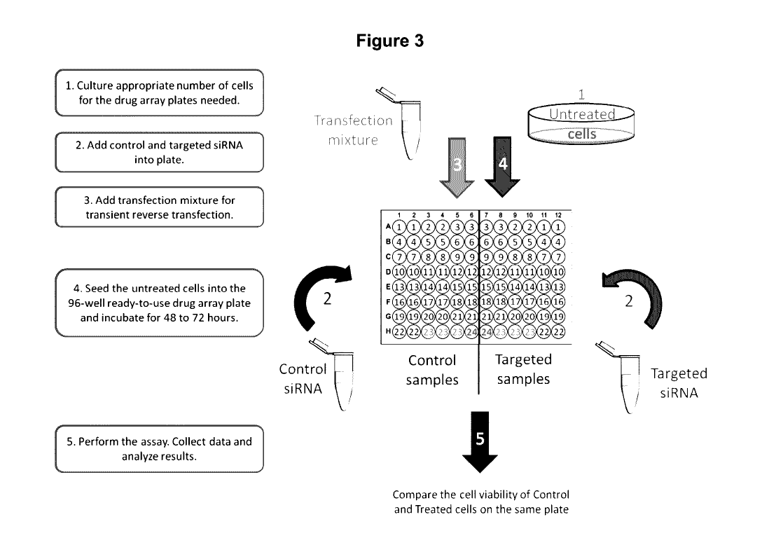

Cells are added to the plates using a repeater pipette to add cells. As shown in FIGS. 1-3, wells #24 (see diagram below) should contain no cells, and only culture media should be added into these wells. The drug arrays are designed in a mirror symmetry layout to cancel out the edge effects. To reduce edge effects, the seeded cells are equilibrated at room temperature for 1 hour before placing the plate into the incubator [7].

### II. Measurement of Viable Cell Number

Various assay kits can be used to measure viable cell numbers. For example, WST-1 assay kit can be used for cell survival, including WST-1 Cell Proliferation Assay Kit (Ser. No. 10/008,883) (Cayman), WST-1 Cell Proliferation Array Kit (KA1384) (Abnova), and Cell Proliferation Reagent WST-1 (05015944001) (Life Science).

WST-1 can be added by using a repeater pipette. The WST-1 should be directly added to the medium and mixed by gentle shaking to avoid WST-1 sticking to the wall. Air bubbles should also be avoided in the wells when reading the plate.

Viable cell numbers can be measured using different readout. For example, luciferase reporter or GFP driven by specific promoter.

### III. Data Analysis and Visualization

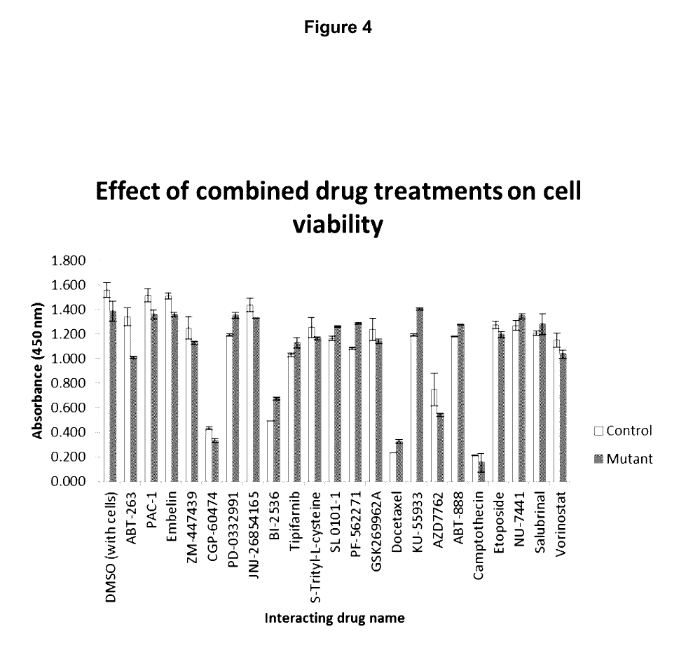

Data is collected into the raw data sheet. The pre-programmed algorithm on the Analysis sheet will subtract the background (plate well #24), normalize against the DMSO control (plate well #23), and calculate the average, standard deviation, inhibition rate and difference in inhibition rates for each treatment and control pair. The results will be transformed into figures, as shown in FIGS. 4-6. Inhibition rate is calculated as “(signal of control−signal of treatment)/signal of control”.

### Examples

### 1. Example of Gene-Drug Interaction Study Using Stable Cell Lines

### Experiment Design

The colorectal cancer cell line HCT116 has a high expression level of a tumor transforming gene. We created a control and mutant gene knockout from this parental cell line, and studied the interaction of this gene with the 22 drugs in Drug Array 1 (Table 1). The control and mutant cells were seeded into the wells on the left and right sides of the plate respectively. Each well contains 6000 cells in 100 μl of culture media. Comparison of the differences in cell viability between the mutant and control can reveal synergism between the gene and drugs.

### Results Interpretation

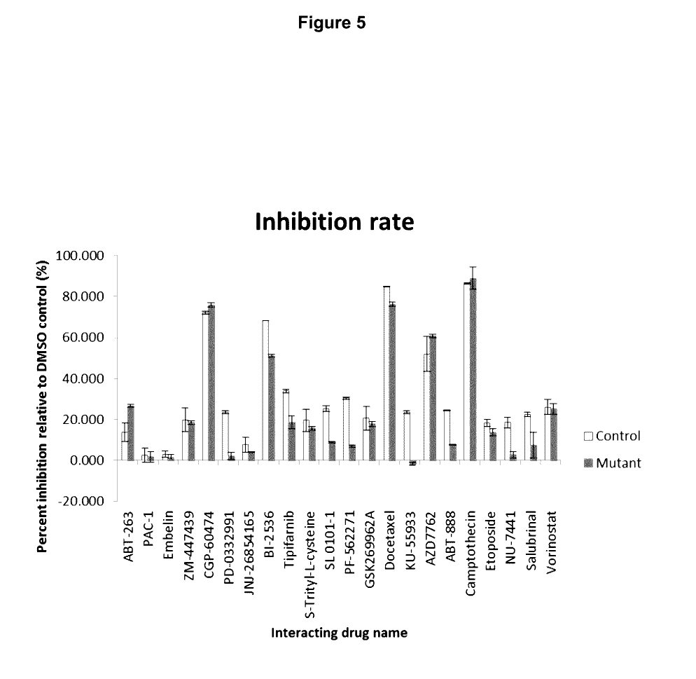

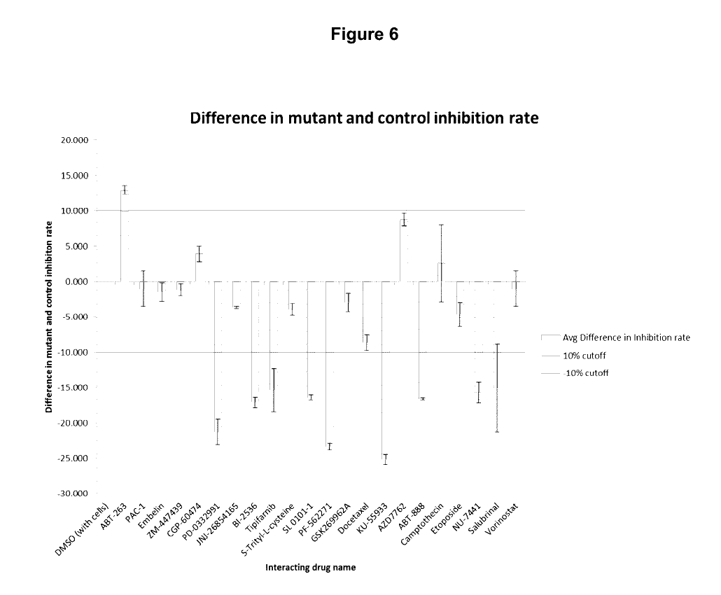

The cell viability is measured as background subtracted Absorbance (FIG. 4), with a higher absorbance reading indicating a higher number of viable cells. For each drug, similar cell viability between the mutant and control indicates that the drug affects both cell lines equally and thus the knockout gene does not affect drug response. If the mutant cells have lower viability than the control, this indicates that the knockout gene sensitizes the cells to the drug. For easier visualization of the differences in viability, the inhibition rate (FIG. 5) and difference of inhibition rates (FIG. 6) are provided. The inhibition rate shows the percent inhibition of cell growth relative to the DMSO control. A greater change in the difference of the mutant and control inhibition rates shows the larger difference in the inhibition rates.

TABLE 1
Drug Array 1: Apoptosis, Cell cycle, Cytoskeleton, DNA damage, HDAC

| Sample | | | |
|---|---|---|---|
| number | Drug Name | Drug Targets | Pathway |
| 1 | ABT-263 | BCL2, BCL-XL, BCL-W | Apoptosis |
| 2 | PAC-1 | CASP3 activator | |
| 3 | Embelin | XIAP | |
| 4 | ZM-447439 | AURKB | Cell cycle |
| 5 | CGP-60474 | CDK1/2/5/7/9 | |
| 6 | PD-0332991 | CDK4/6 | |
| 7 | JNJ-26854165 | MDM2 | |
| 8 | BI-2536 | PLK1/2/3 | |
| 9 | Tipifarnib | Farnesyl-transferase | |
| | | (FNTA) | |
| 10 | S-Trityl-L-cysteine | KIF11 | |
| 11 | SL 0101-1 | RSK, AURKB, PIM3 | |
| 12 | PF-562271 | FAK | Cytoskeleton |
| 13 | GSK269962A | ROCK | |
| 14 | Docetaxel | Microtubules | |
| 15 | KU-55933 | ATM | DNA |
| | | | damage |
| 16 | AZD7762 | CHK1/2 | |
| 17 | ABT-888 | PARP1/2 | |
| 18 | Camptothecin | TOP1 | |
| 19 | Etoposide | TOP2 | |
| 20 | NU-7441 | DNAPK | |
| 21 | Salubrinal | GADD34-PP1C | |
| | | phosphatase | |
| 22 | Vorinostat | HDAC inhibitor Class | HDAC |
| | | I, IIa, IIb, IV | inhibitor |
| 23 | DMSO (with cells) | — | (Control) |
| 24 | DMSO (without cells) | — | |

TABLE 2
Drug Array 2: Kinase signaling pathways (MEK-ERK,
MTOR-S6, NF-κB, NRTK, PAK, PI3K-AKT, PRKC),
NOTCH and TNFα signaling pathways

| Sample | | | |
|---|---|---|---|
| number | Drug Name | Drug Targets | Pathway |
| 1 | AZ628 | BRAF | MEK-ERK |
| 2 | SP 600125 | JNK1,2,3 | |
| 3 | AZD6244 | MEK1/2 | |
| 4 | GW 441756 | NTRK1 | |
| 5 | BIRB 0796 | p38, JNK2 | |
| 6 | CCT007093 | PPM1D | |
| 7 | NSC-87877 | SHP1/2 (PTN6/11) | |
| 8 | Torin 1 | MTOR | MTOR-S6 |
| 9 | PF-4708671 | p70 S6KA | |
| 10 | KIN001-135 | IKKE | NF-KB |
| 11 | Parthenolide | NFKB1 | |
| 12 | BMS-708163 | gamma-secretase | NOTCH |
| 13 | DAPT | g-secretase | |
| 14 | GNF-2 | BCR-ABL | NRTK |
| 15 | BMX-IN-1 | BMX | |
| 16 | BMS-509744 | ITK | |
| 17 | IPA-3 | PAK | PAK |
| 18 | GDC-0068 | AKT1/2/3 | PI3K-AKT |
| 19 | OSU-03012 | PDK1 (PDPK1) | |
| 20 | NVP-BEZ235 | PI3K (Class 1) and | |
| | | mTORC1/2 | |
| 21 | Staurosporine | PRKC | PRKC |
| 22 | Lenalidomide | TNF alpha | TNF alpha |
| 23 | DMSO (with cells) | — | (Control) |
| 24 | DMSO (without cells) | — | |

TABLE 3
Drug Array 3: RacGTPases, Retinoic acid X family, RTK,
SMO, Protein modifications, Metabolism

| Sample | | | |
|---|---|---|---|
| number | Drug Name | Drug Targets | Pathway |
| 1 | QS11 | ARFGAP | RacGTPases |
| 2 | EHT 1864 | RacGTPases | |
| 3 | ATRA | Retinoic acid and RXR | Retinioic acid |
| | | agonist | X family |
| 4 | Dasatinib | ABL, SRC, KIT, PDGFR | RTK |
| 5 | NVP-TAE684 | ALK | |
| 6 | VX-680 | Aurora A/B/C, FLT3, | |
| | | ABL1, JAK2 | |
| 7 | LFM-A13 | BTK | |
| 8 | BIBW2992 | EGFR, ERBB2 | |
| 9 | PD-173074 | FGFR1/3 | |
| 10 | CEP-701 | FLT3, JAK2, NTRK1, RET | |
| 11 | BMS-536924 | IGF1R | |
| 12 | PF-02341066 | MET, ALK | |
| 13 | WH-4-023 | SRC family, ABL | |
| 14 | BAY 61-3606 | SYK | |
| 15 | Pazopanib | VEGFR, PDGFRA, | |
| | | PDGFRB, KIT | |
| 16 | Cyclopamine | SMO | SMO |
| 17 | Elesclomol | HSP70 | Protein |
| 18 | 17-AAG | HSP90 | modification |
| 19 | Bortezomib | Proteasome | |
| 20 | DMOG | Prolyl-4-Hydroxylase | |
| 21 | AICAR | AMPK agonist | Metabolism |
| 22 | Methotrexate | Dihydrofolate reductase | |
| | | (DHFR) | |
| 23 | DMSO | — | (Control) |
| | (with cells) | | |
| 24 | DMSO | — | |
| | (without cells) | | |

### REFERENCES

1. Lord C J, Tutt A N, Ashworth A: Synthetic lethality and cancer therapy: lessons learned from the development of PARP inhibitors. Annual review of medicine 2015, 66:455-470.
2. Misale S, Yaeger R, Hobor S, Scala E, Janakiraman M, Liska D, Valtorta E, Schiavo R, Buscarino M, Siravegna G et al: Emergence of KRAS mutations and acquired resistance to anti-EGFR therapy in colorectal cancer. Nature 2012, 486(7404):532-536.
3. Knickelbein K, Zhang L: Mutant KRAS as a critical determinant of the therapeutic response of colorectal cancer. Genes & Diseases 2015, 2(1):4-12.
4. Kwong L N, Costello J C, Liu H, Jiang S, Helms T L, Langsdorf A E, Jakubosky D, Genovese G, Muller F L, Jeong J H et al: Oncogenic NRAS signaling differentially regulates survival and proliferation in melanoma. Nature medicine 2012, 18(10):1503-1510.
5. Prahallad A, Sun C, Huang S, Di Nicolantonio F, Salazar R, Zecchin D, Beijersbergen R L, Bardelli A, Bernards R: Unresponsiveness of colon cancer to BRAF(V600E) inhibition through feedback activation of EGFR. Nature 2012, 483(7387):100-103.
6. Garnett M J, Edelman E J, Heidorn S J, Greenman C D, Dastur A, Lau K W, Greninger P, Thompson I R, Luo X, Soares J et al: Systematic identification of genomic markers of drug sensitivity in cancer cells. Nature 2012, 483(7391):570-575.
7. Lundholt B K, Scudder K M, Pagliaro L: A simple technique for reducing edge effect in cell-based assays. Journal of biomolecular screening 2003, 8(5):566-570.
   siRNA Array

In another embodiment, the invention further provides a siRNA array that uses siRNAs as tools to survey potential gene-gene or gene-drug interactions. In certain embodiments, the targets of the selected siRNAs include drug targets, significantly mutated genes and transcription factors.

One advantage of siRNA array for drug targets is that siRNAs are usually more specific than most selective chemicals. It therefore serves as a good alternative to Drug Array in screening for potential gene-drug interactions.

Moreover, the siRNA can target non-druggable genes which are also critical in cancer. For example, the significantly mutated genes in cancers identified in large scale Next Generation Sequencing studies [1]. The siRNA array for significantly mutated genes allows user to survey whether these genes are important to the functions of a given gene or drug.

Finally, siRNA array for transcription factors are compiled as the majority of oncogenic signaling pathways converge on sets of transcription factors that ultimately control gene expression patterns resulting in tumor formation and progression as well as metastasis.

One major implication is in screening genes regulating anti-cancer drug resistance. Depletion of the targeted genes therefore sensitizes the cancer cells to anti-cancer drugs, sometimes leading to synthetic lethality. Inducing synthetic lethality in cancer cells is indeed a valuable strategy to selectively kill cancer cells with minimal effect on normal cells. In cancer cells that possess a certain oncogenic activation, the inhibition of a second critical genetic function can cause cell death.

For example, TP53 is an important player in DNA damage signaling pathways. When TP53 is mutated in some tumors, other G2/M checkpoint regulators such as checkpoint kinase 1 (CHK1) compensates for the absence of TP53 and arrests the cell cycle. If both TP53 and CHK1 are inhibited, the tumor cells are unable to arrest cell cycle for DNA damage repair, and thus the accumulation of unrepaired DNA will reduce the viability of tumor cells [2].

As another example, the myelocytomatosis viral oncogene homolog (MYC) is a transcription factor that plays an important role in cell cycle progression, apoptosis, and cellular transformation. Death Receptor 5 (DR5), a member of the TNF-receptor superfamily, is a mediator of apoptosis. MYC is over expressed in some cancers, and it has been shown in fibroblasts that increased expression of MYC causes sensitivity to agonists of DR5 [3].

RNA interference (RNAi) is a useful tool to identify important gene-gene and drug-gene interactions. RNAi based functional genomic screening can lead to the discovery of many drug combinations which may lead to improved anti-cancer treatment, such as MEK inhibitor with CDK4 Inhibitors [4], and RAF Inhibitors with EGFR inhibitors [5].

### Methods

I. General Design

Each siRNA Array plate is designed to compare two different samples in parallel on one ready-to-use 96-well plate. The siRNAs are supplied in a lyophilized form, and each well contains 2.5 pmol of siRNA (final concentration will be 25 nM after adding 100 μl of transfection mixture and cells). It is recommended using co-transfecting two or more siRNAs to avoid reaching an excessively high concentration (>50 nM) which may cause off-target effects and non-specific cytotoxicity. The arrays are designed in a mirror symmetry layout to cancel out the edge effects.

II. Preparation of the Experiments

Determine Cell Number

The cell culture conditions, such as the number of the cells can significantly affect the results and must be empirically determined before the experiment. In general, cells in control wells should be seeded at densities that could reach optimal population densities (80-90% confluence) at the end of the experiment (48-72 hours). Most cancer cells require 2,000-5,000 cells per well in 96 well plates.

The right number of cells is critical to the success of the assay. Too many cells will cause over confluence and deplete the nutrition of the medium before the end of the assay. Too few cells will lead to large variation and increased edge effects.

Determine Drug Concentration for Drug-Gene Interaction

To identify genes promoting resistance to anti-cancer drugs, the given drug should be added in a concentration around IC15-IC50 (a concentration that causes 15%-50% inhibition of cell proliferation).

To identify genes promoting sensitivity to anti-cancer drugs, the given drug should be added in a concentration around IC30-IC75 (a concentration that causes 30%-75% inhibition of cell proliferation). A concentration of IC30-IC50 serves both purposes.

Determine Time Points for Transient Transfection

To observe potential gene-gene interaction, it is preferred to generate isogenic cell lines of targeted genes, for example, stable knockdown/over-expression cell line vs. parent control cell line. Transient transfection may be used as long as the transfection efficiency is higher than 70%. The effect of knock-down of transient transfection usually last for 7 days. The gene-gene interaction experiment should be performed 24-48 hours after transfection.

### Experimental Procedures

### Seeding Cells

### General Protocol for Reverse Transfection

In the siRNA array plates, 2.5 pmol of lyophilized siRNA is provided in each well. For example, Lipofectamine RNAiMAX (Invitrogen) for reverse transfection can be used. First add 25 μl of Opti-MEM into each well and mix to redissolve the siRNA. Next, create a master mix of Lipofectamine and Opti-MEM in an Eppendorf tube. For each well, 0.2 μl of Lipofectamine and 25 μl of Opti-MEM will be needed. Multiply these volumes by the number of wells needed. Incubate the master mix for 15 minutes, then add 25 μl into each well, and mix with the 25 μl of siRNA in the plate. Seed 2000-5000 cells per well in 50 μl of regular media without antibiotics. The final concentration of siRNA in each well will be 25 nM, and final volume will be 100 μl.

### Experiment Design A: Drug-Gene Interaction Study

Prepare the siRNA array for reverse transfection. Add the transfection reagent and Opti-MEM mixture into each well. Cells are suspended and seeded into siRNA Array plate. The drug will be added to form Drug-Gene combination 24 hours after reverse transfection.

### Experiment Design B: Gene-Gene Interaction Study Using Stable Cell Lines

Stable knockdown/over-expression cells and parental control cells are suspended and added to the siRNA array. Incubate the siRNA array in a cell culture incubator for 48-72 hours.

### Experiment Design C: Gene-Gene Interaction Study Using Transient Transfection

To study gene-gene interaction using siRNAs, a mix of siRNAs using the provided siRNA array should be created. The siRNA arrays are then prepared for reverse transfection. Cells are suspended and added into the transfection mix. Incubate the siRNA array in a cell culture incubator for 48-72 hours.

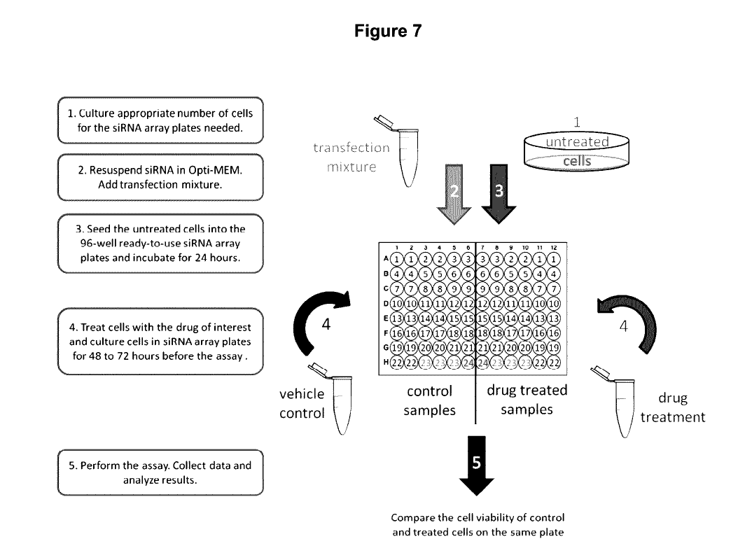

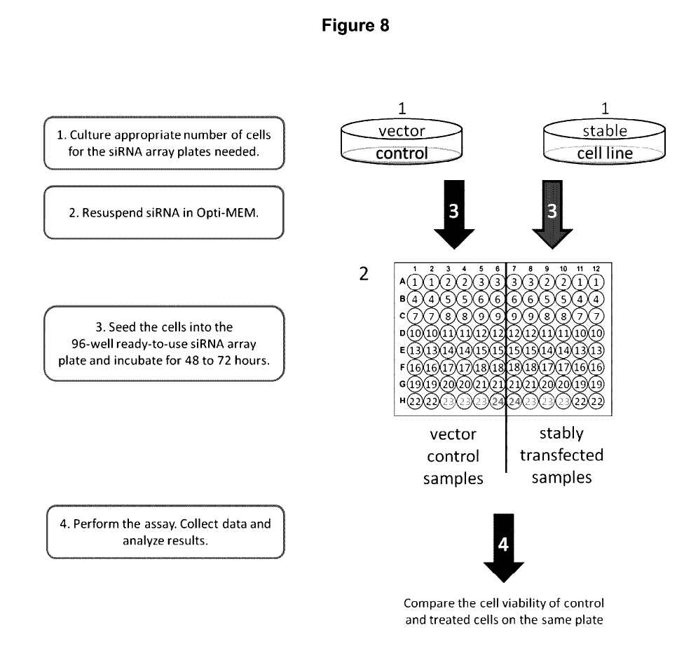

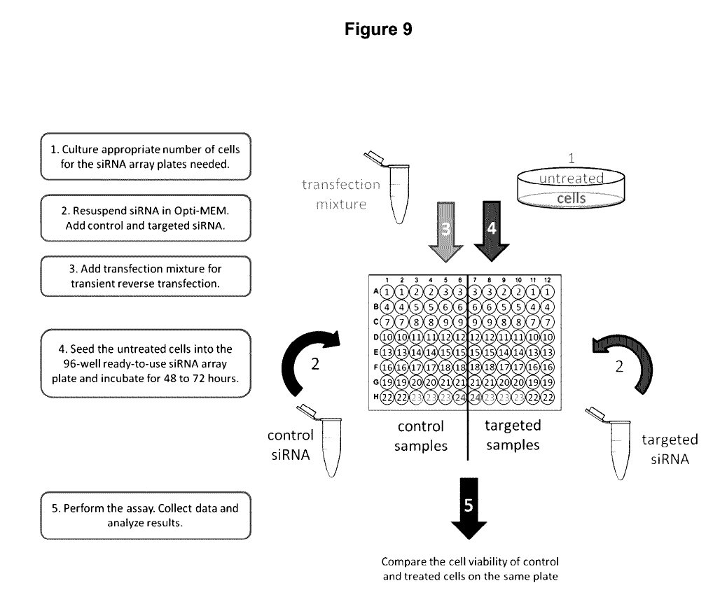

A repeater pipette is used to add cells to the plates. Well #24 (as shown in FIG. 7-9) should contain no cells, and only culture media should be added into these wells.

To reduce edge effects, the seeded cells should be equilibrated at room temperature for 1 hour before placing the plate into the incubator [6].

### Measurement of Viable Cell Number

Various assay kits can be used to measure viable cell numbers. For example, WST-1 assay kit can be used for cell survival, including WST-1 Cell Proliferation Assay Kit (Ser. No. 10/008,883) (Cayman), WST-1 Cell Proliferation Array Kit (KA1384) (Abnova), and Cell Proliferation Reagent WST-1 (05015944001) (Life Science).

WST-1 can be added by using a repeater pipette. The WST-1 should be directly added to the medium and mixed by gentle shaking to avoid WST-1 sticking to the wall. Air bubbles should also be avoided in the wells when reading the plate.

Viable cell numbers can be measured using different readout. For example, luciferase reporter or GFP driven by specific promoter.

### Data Analysis and Visualization

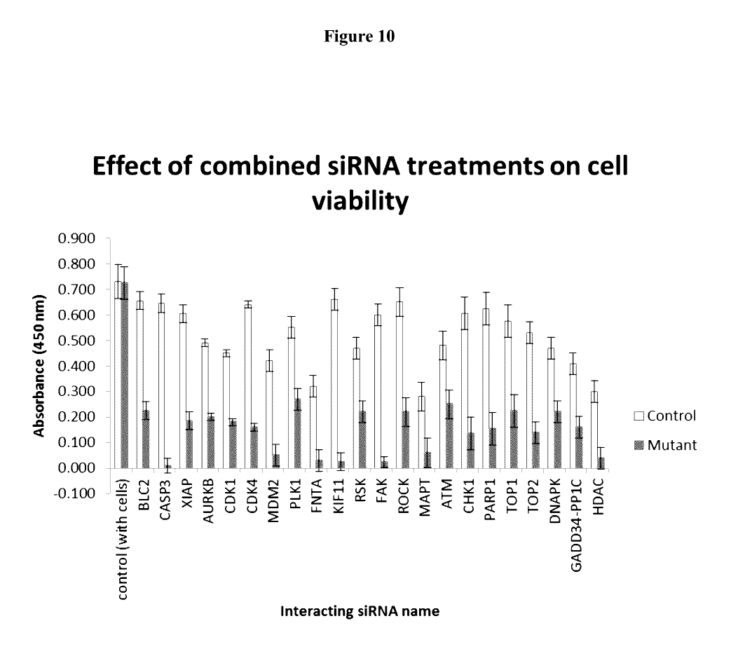

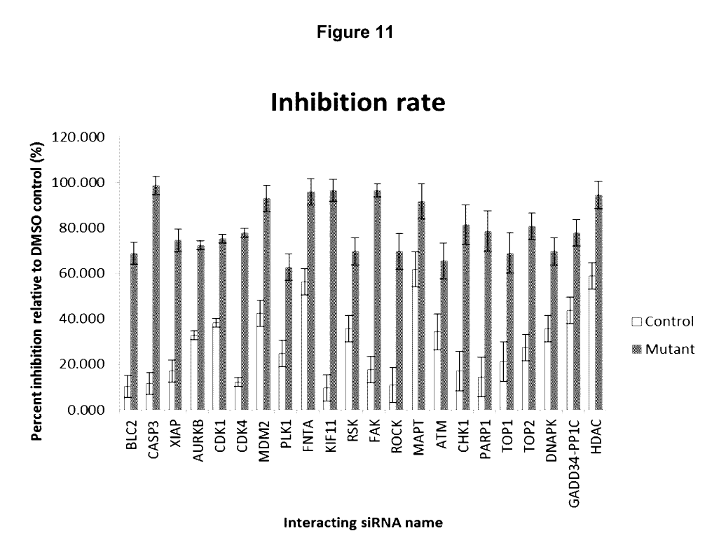

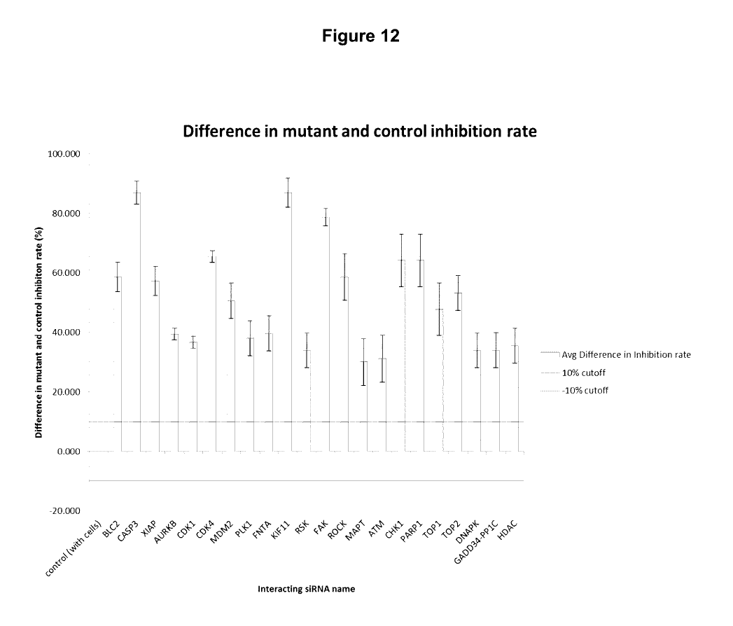

Data is collected into the raw data sheet. The pre-programmed algorithm on the Analysis sheet will subtract the background (plate well #24), normalize against the DMSO control (plate well #23), and calculate the average, standard deviation and inhibition rate and difference in inhibition rates between treatment and control pair. The results will be transformed into figures, as shown in FIGS. 10-12. Inhibition rate is calculated as “(signal of control−signal of treatment)/signal of control”.

TABLE 4
Array 4: Apoptosis, Cell cycle, Cytoskeleton, DNA damage, HDAC

| | siRNA | | |
|---|---|---|---|
| | Target | Gene Title and [Common Name] | Pathway |
| 1 | BCL2 | B-cell CLL/lymphoma 2 | Apoptosis |
| 2 | CASP3 | caspase 3, apoptosis-related | |
| | | cysteine peptidase | |
| 3 | XIAP | X-linked inhibitor of apoptosis, E3 | |
| | | ubiquitin protein ligase | |
| 4 | AURKB | aurora kinase B | Cell cycle |
| 5 | CDK1 | cyclin-dependent kinase 1 | |
| 6 | CDK4 | cyclin-dependent kinase 4 | |
| 7 | MDM2 | MDM2 proto-oncogene, E3 ubiquitin | |
| | | protein ligase | |
| 8 | PLK1 | polo-like kinase 1 | |
| 9 | FNTA | farnesyltransferase, CAAX | |
| | box, alpha | | |
| 10 | KIF11 | kinesin family member 11 | |
| 11 | RPS6KA3 | ribosomal protein S6 kinase, 90 kDa, | |
| | | polypeptide 3 | |
| 12 | PTK2 | protein tyrosine kinase 2 [FAK] | Cytoskeleton |
| 13 | ROCK1 | Rho-associated, coiled-coil | |
| | | containing protein kinase 1 | |
| 14 | MAPT | microtubule-associated protein tau | |
| 15 | ATM | ATM serine/threonine kinase | DNA damage |
| 16 | CHEK1 | checkpoint kinase 1 | |
| 17 | PARP1 | poly (ADP-ribose) polymerase 1 | |
| 18 | TOP1 | topoisomerase (DNA) I | |
| 19 | TOP2A | topoisomerase (DNA) II alpha | |
| 20 | PRKDC | protein kinase, DNA-activated, | |
| | | catalytic polypeptide [DNAPK] | |
| 21 | PPP1R15A | protein phosphatase 1, regulatory | |
| | | subunit 15A [GADD34] | |
| 22 | HDAC | histone deacetylase | HDAC |
| | | | inhibitor |
| 23 | Scrambled | | (Control) |
| | siRNA | | |
| 24 | Background | | |
| | (without | | |
| | cells) | | |

TABLE 5
Array 5: Kinase signaling pathways (MEK-ERK, MTOR-S6, NF-κB, NRTK, PAK,
PI3K-AKT, PRKC), NOTCH and TNFα signaling pathways

| | siRNA Target | Gene Title and [Common Name] | Pathway |
|---|---|---|---|
| 1 | BRAF | B-Raf proto-oncogene, serine/threonine kinase | MEK-ERK |
| 2 | MAPK8 | mitogen-activated protein kinase 8 [JNK1] | |
| 3 | MAP2K1 | mitogen-activated protein kinase kinase 1 [MEK1] | |
| 4 | NTRK1 | neurotrophic tyrosine kinase, receptor, type 1 | |
| 5 | MAPK1 | mitogen-activated protein kinase 1 [p38] | |
| 6 | PPM1D | protein phosphatase, Mg2+/Mn2+ dependent, 1D | |
| 7 | PTPN6 | protein tyrosine phosphatase, non-receptor type | |
| | | 6 [SHP1] | |
| 8 | MTOR | mechanistic target of rapamycin | MTOR-S6 |
| | | (serine/threonine kinase) | |
| 9 | RPS6KB1 | ribosomal protein S6 kinase, 70 kDa, polypeptide 1 | |
| | | [p70 S6KA] | |
| 10 | IKBKE | inhibitor of kappa light polypeptide gene | NF-KB |
| | | enhancer in B-cells, kinase epsilon [IKKE] | |
| 11 | NFKB1 | nuclear factor of kappa light polypeptide gene | |
| | | enhancer in B-cells 1 | |
| 12 | APH1A | APH1A gamma secretase subunit | NOTCH |
| 13 | PSENEN | presenilin enhancer gamma secretase subunit | |
| 14 | BCR-ABL | breakpoint cluster region/ABL proto-oncogene 1, | NRTK |
| | | non-receptor tyrosine kinase | |
| 15 | BMX | BMX non-receptor tyrosine kinase | |
| 16 | ITK | IL2-inducible T-cell kinase | |
| 17 | PAK1 | p21 protein (Cdc42/Rac)-activated kinase 1 | PAK |
| 18 | AKT1 | v-akt murine thymoma viral oncogene homolog 1 | PI3K-AKT |
| 19 | PDK1 | 3-phosphoinositide dependent protein kinase 1 | |
| 20 | PIK3CA | phosphatidylinositol-4,5-bisphosphate 3-kinase, | |
| | | catalytic subunit alpha | |
| 21 | PRKCA | protein kinase C, alpha | PRKC |
| 22 | TNF | tumor necrosis factor | TNF alpha |
| 23 | Scrambled siRNA | | (Control) |
| 24 | Background | | |
| | (without cells) | | |

TABLE 6
Array 6: RacGTPases, Retinoic acid X family, RTK, SMO, Protein
modifications, Metabolism

| | siRNA | | |
|---|---|---|---|
| | Target | Gene Title and [Common Name] | Pathway |
| 1 | ARFGAP1 | ADP-ribosylation factor GTPase | RacGTPases |
| | | activating protein 1 | |
| 2 | RAC1 | ras-related C3 botulinum toxin substrate | |
| | | 1 (rho family, small GTP binding | |
| | | protein Rac1) | |
| 3 | RXRA | retinoid X receptor, alpha | Retinioic |
| | | | acid |
| | | | X family |
| 4 | ABL1 | ABL proto-oncogene 1, non-receptor | RTK |
| | | tyrosine kinase | |
| 5 | ALK | anaplastic lymphoma receptor tyrosine | |
| | | kinase | |
| 6 | AURKA | aurora kinase A | |
| 7 | BTK | Brutonagammaglobulinemia tyrosine | |
| | | kinase | |
| 8 | EGFR | epidermal growth factor receptor | |
| 9 | FGFR1 | fibroblast growth factor receptor 1 | |
| 10 | FLT3 | fms-related tyrosine kinase 3 | |
| 11 | IGF1R | insulin-like growth factor 1 receptor | |
| 12 | MET | MET proto-oncogene, receptor tyrosine | |
| | | kinase | |
| 13 | SRC | SRC proto-oncogene, non-receptor | |
| | | tyrosine kinase | |
| 14 | SYK | spleen tyrosine kinase | |
| 15 | KDR | kinase insert domain receptor | |
| | | [VEGFR2] | |
| 16 | SMO | smoothened, frizzled class receptor | SMO |
| 17 | HSPA4 | heat shock 70 kDa protein 4 [HSP70] | Protein |
| 18 | HSP90AA1 | heat shock protein 90 kDa alpha | modification |
| | | (cytosolic), class A member 1 [HSP90] | |
| 19 | PSMC5 | proteasome 26S subunit, ATPase 5 | |
| 20 | P4HA1 | prolyl 4-hydroxylase, alpha | |
| | | polypeptide I | |
| 21 | PRKAA1 | protein kinase, AMP-activated, alpha 1 | Metabolism |
| | | catalytic subunit [AMPK] | |
| 22 | DHFR | dihydrofolate reductase | |
| 23 | Scrambled | | (Control) |
| | siRNA | | |
| 24 | Background | | |
| | (without | | |
| | cells) | | |

### REFERENCES

1. Lawrence, M. S., et al., Discovery and saturation analysis of cancer genes across 21 tumour types. Nature, 2014. 505(7484): p. 495-501.
2. Morandell, S. and M. B. Yaffe, Exploiting synthetic lethal interactions between DNA damage signaling, checkpoint control, and p53 for targeted cancer therapy. Prog Mol Biol Transl Sci, 2012. 110: p. 289-314.
3. Wang, Y, et al., Synthetic lethal targeting of MYC by activation of the DR5 death receptor pathway. Cancer Cell, 2004. 5(5): p. 501-12.
4. Kwong, L. N., et al., Oncogenic NRAS signaling differentially regulates survival and proliferation in melanoma. Nat Med, 2012. 18(10): p. 1503-10.
5. Prahallad, A., et al., Unresponsiveness of colon cancer to BRAF(V600E) inhibition through feedback activation of EGFR. Nature, 2012. 483(7387): p. 100-3.
6. Lundholt, B. K., K. M. Scudder, and L. Pagliaro, A simple technique for reducing edge effect in cell-based assays. J Biomol Screen, 2003. 8(5): p. 566-70.

1-26. (canceled)

27. A method of identifying drug-drug, gene-drug or gene-gene interaction comprising:
a) providing a plurality of arrays, each array including a plurality of ready-to-use plate wells, and each well including a bioactive material to target one or more cell component;
b) adding a control and a candidate agent into the wells to form a control-material mix and an agent-material mix, respectively;
c) when the control and the candidate agent are not cells, culturing a predetermined number of cells according to the number of the arrays and suspending and plating the cells into the arrays, when the control and the candidate agent are cells, no additional cells are needed;
d) incubating the arrays in a cell culture incubator;
e) measuring a predetermined signal; and
f) collecting and analyzing data, thereby identifying the drug-drug, gene-drug, or gene-gene interaction.

28. The method of claim 27, wherein the predetermined signal is viable cell numbers in each well.

29. The method of claim 27, wherein the candidate agent is a transfected/treated cell, a stable knockdown/over-expression cell, a genetically modified cell, a chemical, a drug, an siRNA, an miRNA, a growth factor, a hormone, a proteins, or a bioactive agent.

30. The method of claim 27, wherein the bioactive material is a drug or siRNA.

31. The method of claim 27, wherein the bioactive material in each well is provided at a concentration that is optimized to reduce or enhance the activities of corresponding targets or functions in the cells.

32. The method of claim 30, wherein the drug in each well is provided at a concentration of 0.5 μM or 2.5 μM.

33. The method of claim 27, wherein the arrays are designed in mirror or rotational symmetry to cancel out variations.

34. The methods of claim 27, wherein the cells are equilibrated before incubating to remove the edge effects.

35. The method of claim 27, wherein the arrays are incubated for 48-72 hours.

36. The method of claim 29, wherein when the candidate agent is a transfected/treated cell, a stable knockdown/over-expression cell, or a genetically modified cell and the control is a cell, the candidate agent and control are cultured, suspended and plated into the arrays.

37. The method of claim 27, wherein when the bioactive material is a drug, the candidate agent is provided at a concentration that causes 15%-30% inhibition of cell proliferation.

38. The method of claim 27, wherein when the bioactive material is an siRNA, the candidate agent is provided at a concentration that causes 15%-50% inhibition of cell proliferation to identify genes promoting resistance to anti-cancer drugs and at a concentration that causes 30%-75% inhibition of cell proliferation to identify genes promoting sensitivity to anti-cancer drugs.

39. An array for identifying drug-drug, gene-drug or gene-gene interaction comprising:
a plurality of ready-to-use plate wells, and
a bioactive material being included in each well,
wherein a control and a candidate agent are added into the wells to form a control-material mix and an agent-material mix, respectively; when the control and candidate agent are not cells, a predetermined number of cells are cultured, suspended and plated in the array, when the control and candidate agent are cells, no additional cells are needed; the array is incubated in a cell culture incubator; a predetermined signal is measured; and data is collected and analyzed to identify the drug-drug, gene-drug, or gene-gene interaction.

40. The array of claim 39, wherein the bioactive material is a drug or siRNA.

41. The array of claim 39, wherein the predetermined signal is viable cell numbers in each well.

42. The array of claim 39, wherein the candidate agent is a transfected/treated cell, a stable knockdown/over-expression cell, a genetically modified cell, a chemical, a drug, an siRNA, an miRNA, a growth factor, a hormone, a proteins, or a bioactive agent.

43. The array of claim 39, wherein the bioactive material in each well is provided at a concentration that is optimized to reduce or enhance the activities of corresponding targets or functions in the cells.

44. The array of claim 40, wherein the drug in each well has a concentration of 0.5 μM or 2.5 μM.

45. The array of claim 39, wherein when the bioactive material is a drug, the candidate agent is provided at a concentration that causes 15%-30% inhibition of cell proliferation.

46. The array of claim 39, wherein when the bioactive material is an siRNA, the candidate agent is provided at a concentration that causes 15%-50% inhibition of cell proliferation to identify genes promoting resistance to anti-cancer drugs and at a concentration that causes 30%-75% inhibition of cell proliferation to identify genes promoting sensitivity to anti-cancer drugs.
